(19) United States
(12) Patent Application Publication
(10) Pub. No.: US 2012/0065430 A1
(43) Pub. Date: Mar. 15, 2012

(54) Application of Metal Amidoborane and Their Derivatives in Reduction Reaction

(76) Inventors: Ping Chen, Dalian (CN); Weiliang Xu, Dalian (CN); Zhitao Xiong, Dalian (CN); Guotao Wu, Dalian (CN)

(21) Appl. No.: 12/952,518

(22) Filed: Nov. 23, 2010

(30) Foreign Application Priority Data
Sep. 10, 2010 (CN) 201010278024.5

(51) Int. Cl. C07C 211/43; C07C 33/00

(52) U.S. Cl. 564/305; 568/807

(57) ABSTRACT

Polarized unsaturated functional groups is directly reduced by using metal amidoborane or derivatives thereof through double hydrogen transfer process. Over 99% conversion of reagents and high isolated yield of products can be achieved after reaction.

### FIELD OF THE INVENTION

This invention relates to a method of reducing polarized unsaturated functional groups by using metal amidoboranes and their derivatives.

### BACKGROUND OF THE INVENTION

Metal amidoboranes and their derivatives are attractive new materials for chemical hydrogen storage. Because of co-exist of hydridic B—H and protic N—H bonds, the materials can release hydrogen below 100° C. without the assistance of catalysis.

Transfer hydrogenation is a useful method to reduce unsaturated functional group. Hydrogen donor, DH₂, as a reducing reagent, is utilized in the process. The hydrogens of the donor are nonequivalent and transfer sequentially to unsaturated functional group. Different kind of hydrogen will transfer from hydrogen donor to unsaturated functional group in the reduction process to realize the purpose.

Ammonia borane (NH₃BH₃)has been reported to reduce imines by double hydrogen transfer process. However, the reducing abilities of metal amidoborane compounds have not been studied yet.

### SUMMARY OF THE INVENTION

The invention is related to the process of reducing organic compound having polarized unsaturated functional groups by metal amidoboranes and their derivatives, wherein the organic compound having the formula which include R₁—C(=X₁)—R₂, R₃—C(R₄)=N—R₅, R₆—C≡N, wherein R₁, R₂, R₃, R₄, R₅, and R₆ are selected from the group consisting of hydrogen, substituted or unsubsituted alkyl, aryl, amine and alkoxyl groups; X1 is selected from O or S; metal amidoborane has a formula of M(NH₂BH₃)ₓ, wherein x is valence of M, M is at least one metal elements selected from the group 1 to 13 (standard period table, IUPAC system), the metal amidoborane is preferable selected from the group consisting of LiNH₂BH₃, NaNH₂BH₃, KNH₂BH₃, Mg(NH₂BH₃)₂, Ca(NH₂BH₃)₂, Sr(NH₂BH₃)₂ and ternary or multinary amidobrane such as Li₂Mg(NH₂BH₃)₄ etc. The its derivative has a formula of X₂—nY, wherein X₂ is the metal amidoborane and Y is the ligand such as ammonia borane, NH₃, THF, etc which can form complex with metal amidoborane or ammonia borane, n is the number of ligand. The derivative is selected from the group consisting of LiNH₂BH₃.NH₃BH₃, NaNH₂BH₃.NH₃BH₃, KNH₂BH₃.NH₃BH₃, RbNH₂BH₃.NH₃BH₃, CsNH₂BH₃.NH₃BH₃, Be(NH₂BH₃).NH₃, Mg(NH₂BH₃).NH₃, Ca(NH₂BH₃).NH₃, Sr(NH₂BH₃).NH₃ and Ba(NH₂BH₃).NH₃.

Preferably, the derivative is selected from the group consisting of LiNH₂BH₃.NH₃BH₃, NaNH₂BH₃.NH₃BH₃, Mg(NH₂BH₃)₂.NH₃ and Ca(NH₂BH₃)₂.NH₃ or mixture thereof.

The organic compound having polarized unsaturated functional groups is R₁—C(=O)—R₂ or R₃—C(R₄)=N—R₅ , wherein R₁ and R₂ are selected from substituted and unsubsituted alkyl, or substituted and unsubsituted aryl; R₄ is selected from the group consisting of hydrogen, substituted and unsubsituted alkyl, substituted and unsubsituted aryl groups; R₃ and R₅ are substituted aryl or unsubsituted aryl.

When R₁, R₂, R₃, R₄, R₅ and R₆ are selected from substituted and unsubsituted alkyl, or substituted and unsubsituted alkoxyl, the carbon atoms is less than 10; when R₁, R2, R₃, R₄, R₅ and R₆ are selected from substituted and unsubsituted aryl, the carbon atoms is less than 60.

Alkyl is selected from the group consisting of methyl, ethyl, propyl, isopropyl, butyl, s-butyl, isobutyl, pentyl, neopentyl, hexyl, heptyl, isoheptyl, 2-ethylhexyl, cyclohexyl and octyl. Alkoxyl is selected from the group consisting of methoxyl, ethoxyl, propoxyl, isopropoxyl, butoxyl, s-butoxyl, isobutoxyl, pentoxyl, neopentoxyl, hexoxyl, heptoxyl, isoheptoxy, 2-ethylhexoxyl, or octoxyl.

Aryl is selected from phenyl, biphenyl, naphthyl, anthryl, phenanthryl, and heterocyclic compound having aromaticity which is select from furyl, thienyl, pyrryl, oxazolyl, thiazolyl, imidazolyl, pyrazolyl, or pyridyl.

“substituted” refers to the addition of one or more substituted groups to a compound or functional group. Substituent groups in the invention do not cause the compounds to be unstable or unsuitable for use in an intended reaction and are inert under reaction conditions.

The substituent groups are selected from the group consisting of alkyl, alkoxyl, halogen, or nitrato.

Compounds for formula R₁—C(=O)—R₂ are one or more compounds selected from the group consisting of cyclohexanone, acetophenone 4-methylacetophenone, 4-methoxyacetophenone, 4-nitroacetophenone, benzylacetone, 4-phenyl-3-buten-2-one, benzophenone and 4-chloroacetophenone.

Compounds for formula R₃—C(R₄)=N—R₅ are one or more compounds selected the group consisting of N-benzylideneaniline, N-(4-methylbenzylidene)aniline, 4-methyl-N-benzylidene aniline, N-(4-chlorobenzylidene)aniline, N-(4-methoxybenzylidene)aniline, and N-(4-nitrobenzylidene)aniline.

The temperature for the invention is in the rang of 20° C. to 60° C. .

The suitable solvent is needed in the process of the invention, which is selected from the group consisting of THF, glyme, diglyme, triglyme, crown ether, dichloromethane and dioxane.

The molar ratio of metal amidobrane or derivatives to polarized unsaturated functional group is 1:1 to 1:2.

The compounds with the formula R₁—C(=O)—R₂, to become corresponding secondary alcohol after reaction, and compounds with the formula R₃—C(R₄)=N—R₅, to become corresponding secondary amine in the process.

Y is the ligand selected from the group consisting of NH₃BH₃, NH₃, pyridine, quadrol, triphenylphosphine or THF.

Comparing to the traditional boron hydride (such as LiBH₄), the metal amidoboranes and their derivatives having positively charged atom of hydrogen and electronegative atom of hydrogen which can transfer to the unsaturated functional group simultaneously in the reaction can avoid the steps of hydrolysis and solvolysis in traditional boron hydride reaction, and can be more convenient than the traditional method. Meanwhile, the metal amidoboranes and their derivatives have more reactivity than other reagent used in double hydrogen transfer reactions. For example, it is reported in the literature that amidoboranes (NH₃BH₃) reducing N-benzylideneaniline (imine) in the reaction with THF as reagent at the temperature of 60° C., the proportion of materials is 1:1, the reaction time is 7 hs, while it takes 2 hs in the reaction of lithium amidoboranes (LiNH₂BH₃) reducing N-benzylideneaniline with proportion of materials being 1:1 at the room temperature, the conversion rate is more than 99%.

Amidoboranes and their derivatives can reduce unsaturated organic polar functional groups directly by double hydrogen transfer reactions, the conversion of reactants can be over 99% after the reaction, and the product of higher yield can be obtained.

### BRIEF DESCRIPTION OF THE DRAWINGS

FIG. 1. In-situ FTIR for reaction between LiNH3BH3 and benzophenone at room temperature,

FIG. 2. In-situ FTIR for reaction between LiNH₃BH₃ and N-benzylideneaniline at room temperature.

### DESCRIPTION OF THE PREFERRED EMBODIMENT

### Example 1

Reducing benzophenone into α-phenylbenzenemethanol with LiNH₂BH₃ 5 ml solution of 0.1 M LiNH₂BH₃ in THF was slowly added into 1 ml solution of 1 M benzophenone in THF under room temperature in a closed glass bottle. FT—IR spectrometer was used to observe the consumption of carbonyl group and formation of OH group. Stirring the solution and finish the reaction after the reaction was stopped one hour later. Analysis indicated conversion of benzophenone is over more than 99%. THF was evaporated by rotary, then 10 ml hexane was added into the glass bottle to extract residues alcohol formed for two times. Then, clear hexane solution was collected after centrifugation. Next, hexane was removed with the rotary evaporation evaporated to leave rude product. In the end, further column chromotrography was utilized to purify alcohol rude product to obtain the end product. The isolated yield of α-phenylbenzenemethanol is 94%.

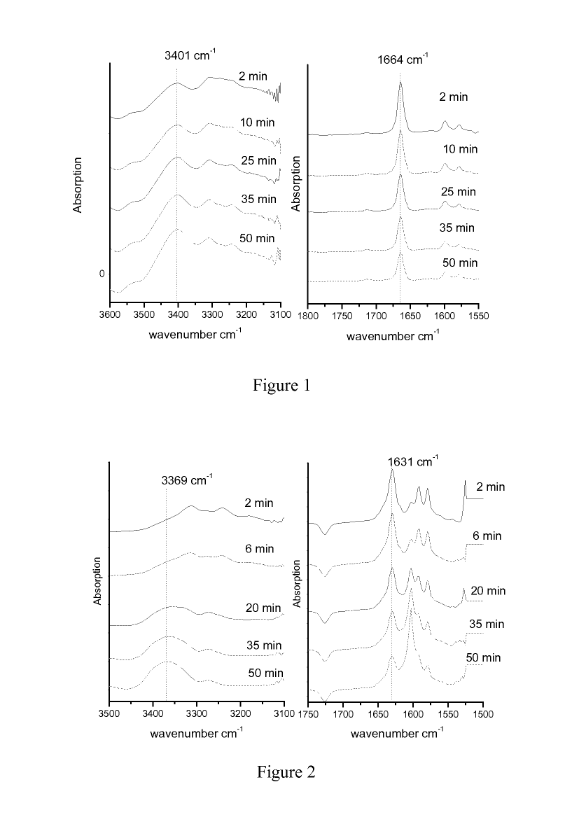

As can be seen from the FIG. 1 that In-situ FTIR show that during the reaction, absorption of C=0 stretch vibration at 1664 cm⁻¹ was decreasing accompanied with increasing absorption of OH stretch vibration at 3401 cm⁻¹during the reaction.

Characteristic data of product(see FIG. 1): ¹H NMR (500 MHz, CDCl₃): δ (ppm) 2.22-2.23 (d, J=3.58 Hz, O—H), 5.73-5.74 (d, J=3.42 Hz, 1H), 7.15-7.30 (m, 10H); 13C NMR (500 MHz, CDCl₃) : δ (ppm) 76.29, 126.55, 127.58, 128.50, 143.82; FT—IR (neat): 3259, 1017 cm⁻¹.

### Example 2

Reducing N-benzylideneaniline into N-benzylaniline with LiNH₂BH₃

5 ml solution of 0.1 M LiNH₂BH₃ in THF was slowly added into lml solution of 0.5 M N-benzylideneaniline in THF under room temperature in a closed glass bottle. FTIR spectrometer was used to observe the consumption of imine group and formation of amine group. The reaction was stopped two hours later. Analysis indicated conversion of N-benzylideneaniline is over more than 99%. THF was evaporated by rotary, then 10 ml hexane was added into the glass bottle to extract amine formed residues for two times. Then, clear hexane solution was collected after centrifugation. Next, hexane was removed with the rotary evaporation evaporated to leave rude product. In the end, further column chromotrography was utilized to purify rude amine product to obtain the end product. The isolated yield of N-benzylaniline is more than 93%. In-situ FTIR (in FIG. 2) show that during the reaction, absorption of C=N stretch vibration at 1631 cm⁻¹ was decreasing accompanied with increasing absorption of NH stretch vibration at 3369 cm⁻¹. (see FIG. 2) Characteristic data of product: ¹H NMR (500 MHz, CDCl₃): δ≢(ppm) 4.05 (s, N—H), 4.36 (s, 2H), 6.67-6.78 (m, 3H), 7.20-7.42 (m, 7H); ¹³C NMR (500 MHz, CDC13): δ (ppm) 48.31, 112.84, 117.55, 127.19, 127.48, 128.60, 129.23, 139.45, 148.15;FT—IR (neat): 3419, 2920, 1602, 1505, 750 cm⁻¹.

### Example 3

Reducing benzophenone into α-phenylbenzenemethanol with Ca(NH₂BH₃)₂

5 ml solution of 0.1 M Ca(NH₂BH₃)₂ in THF was slowly added into 1 ml solution of 1M benzophenone in THF under room temperature in a closed glass bottle. FTIR spectrometer was used to observe the consumption of carbonyl group and formation of OH group. The reaction was stopped one hour later. Analysis indicated conversion of benzophenone is more than over 99%. THF was evaporated by rotary, then 10 ml hexane was added into the glass bottle to extract alcohol formed for two times. Then, clear hexane solution was collected after centrifugation. Next, hexane was evaporated to leave rude product. In the end, further column chromotrography was utilized to purify alcohol product. The isolated yield of α-phenylbenzenemethanol is 90%.

Characteristic data of product: ¹H NMR (500 MHz, CDCl₃): δ (ppm) 2.22-2.23 (d, J=3.58 Hz, O—H), 5.73-5.74 (d, J=3.42 Hz, 1H), 7.15-7.30 (m, 10H); ¹³C NMR (500 MHz, CDCl₃) : δ (ppm) 76.29, 126.55, 127.58, 128.50, 143.82; FT—IR (neat): 3259, 1017 cm⁻¹.

### Example 4

Reducing N-benzylideneaniline into N-benzylaniline with Ca(NH₂BH₃)₂

5 ml solution of 0.1 M Ca(NH₂BH₃)₂ in THF was slowly added into lml solution of 0.5 M N-benzylideneaniline in THF under room temperature in a closed glass bottle. FTIR spectrometer was used to observe the consumption of imine group and formation of amine group. The reaction was stopped two hours later. Analysis indicated over 99% conversion of N-benzylideneaniline. THF was evaporated, then 10 ml hexane was added into the glass bottle to extract amine formed for two times. Then, clear hexane solution was collected after centrifugation. Next, hexane was evaporated to leave liquid residue. In the end, further column chromotrography was utilized to purify amine product. The isolated yield of N-benzylaniline is 87%.

Characteristic data of product: ¹H NMR (500 MHz, CDCl₃): δ (ppm) 4.05 (s, N—H), 4.36 (s, 2H), 6.67-6.78 (m, 3H), 7.20-7.42 (m, 7H); ¹³C NMR (500 MHz, CDCl₃): δ (ppm) 48.31, 112.84, 117.55, 127.19, 127.48, 128.60, 129.23, 139.45, 148.15; FT—IR (neat): 3419, 2920, 1602, 1505, 750 cm⁻¹.

### Example 5

Reducing benzophenone into α-phenylbenzenemethanol with LiNH₂BH₃.NH₃BH₃

5 ml solution of 0.1 M LiNH₂BH₃.NH₃BH₃ in THF was slowly added into lml solution of 1M benzophenone in THF under room temperature in a closed glass bottle. FTIR spectrometer was used to observe the consumption of carbonyl group and formation of OH group. The reaction was stopped one hour later. Analysis indicated over 99% conversion of benzophenone. THF was evaporated, then 10 ml hexane was added in the glass bottle to extract alcohol formed for two times. Then, clear hexane solution was collected after centrifugation. Next, hexane was evaporated to leave rude product. In the end, further column chromotrography was utilized to purify rude product. The isolated yield of α-phenylbenzenemethanol is 95%.

Characteristic data of product: ¹H NMR (500 MHz, CDCl₃): δ (ppm) 2.22-2.23 (d, J=3.58 Hz, O—H), 5.73-5.74 (d, J=3.42 Hz, 1H), 7.15-7.30 (m, 10H); ¹³C NMR (500 MHz, CDCl₃) : δ (ppm) 76.29, 126.55, 127.58, 128.50, 143.82; FT—IR (neat): 3259, 1017 cm⁻¹.

### Example 6

Reducing N-benzylidene aniline into N-benzylaniline with LiNH₂BH₃.NH₃BH₃

5 ml solution of 0.1 M LiNH₂BH₃.NH₃BH₃ in THF was added into 1 ml solution of 0.5 M N-benzylideneaniline in THF under room temperature in a closed glass bottle. FTIR spectrometer was used to observe the consumption of imine group and formation of amine group. The reaction was stopped two hours later. Analysis indicated over 99% conversion of N-benzylideneaniline. THF was evaporated, then 10 ml hexane was added into the glass bottle to extract amine formed for two times. Then, clear hexane solution was collected after centrifugation. Next, hexane was evaporated to leave rude product. In the end, further column chromotrography was utilized to purify rude product. The isolated yield of N-benzylaniline is 93%.

Characteristic data of product: ¹H NMR (500 MHz, CDCl₃): δ (ppm) 4.05 (s, N—H), 4.36 (s, 2H), 6.67-6.78 (m, 3H), 7.20-7.42 (m, 7H); ¹³C NMR (500 MHz, CDC13): δ (ppm) 48.31, 112.84, 117.55, 127.19, 127.48, 128.60, 129.23, 139.45, 148.15; FT—IR (neat): 3419, 2920, 1602, 1505, 750 cm⁻¹.

1. A process of reducing compounds with polarized unsaturated functional group by metal amidoborane and their derivatives, the said compounds with polarized unsaturated functional group have the formula R₁—C(=X₁)—R₂, R₃—C(R₄)=N—R₅, R₆—C≡N,
wherein R₁, R₂, R₃, R₄, R₅ and R₆ are selected from the group consisting of hydrogen, substituted or unsubstituted alkyl, aryl, amine and alkoxyl groups;
X₁ is selected from O and S;
the metal amidoborane has a formula of M(NH₂BH₃)ₓ, x is valence of M chosen from 1-8, M is 1 to 3 metal elements selected from the group 1 to 13 of standard period table;
the derivative has a formula of X₂—nY, wherein X₂ is the said metal amidoborane
and Y is ligand, n is the number of ligand chosen from 1-8.

2. The process according to claim 1, wherein M in M(NH₂BH₃)ₓ is 1 to 3 metal elements selected from Li, Na, K, Rd, Cs, Mg, Ca, Sr, Ba, Al, Ti, V, Cr, Mn, Fe, Co, Ni, Cu and Zn.

3. The process according to claim 1, wherein the derivative is selected from LiNH₂BH₃.NH₃BH₃, NaNH₂BH₃.NH₃BH₃, KNH₂BH₃.NH₃BH₃, RbNH₂BH₃.NH₃BH₃, CsNH₂BH₃.NH₃BH₃, Be(NH₂BH₃).NH₃, Mg(NH₂BH₃).NH₃, Ca(NH₂BH₃).NH₃, Sr(NH₂BH₃).NH₃ and Ba(NH₂BH₃).NH₃.

4. The process according to claim 2, wherein the metal amidoborane is selected from LiNH₂BH₃, NaNH₂BH₃, KNH₂BH₃, Ca(NH₂BH₃)₂ and Li₂Mg(NH₂BH₃)₄; the derivative is selected from LiNH₂BH₃.NH₃BH₃, NaNH₂BH₃.NH₃BH₃, KNH₂BH₃.NH₃BH₃, Mg(NH₂BH₃)₂.NH₃ , Ca(NH₂BH₃)₂.NH₃, and a mixture thereof.

5. The process according to claim 1, the formulas of polarized unsaturated functional group for R₁—C(=O)—R₂ and R₃—C(R₄)=N—R₅, wherein R₁ and R₂ are selected from the group consisting of substituted and unsubstituted alkyl, or aryl groups; R₄ is selected from the group consisting of hydrogen, substituted and unsubstituted alkyl, or aryl groups; R₃ and R₅ are selected from the group consisting of substituted and unsubstituted aryl group.

6. The process according to claim 1, wherein R₁, R₂, R₃, R₄, R₅ and R₆ are substituted and unsubstituted alkyl or substituted and unsubstituted alkoxyl up to 10 carbon atoms; R₁, R₂, R₃, R₄, R₅ and R₆ are substituted and unsubstituted aryl groups less than 60 carbon atoms
the alkyl groups is selected from the group consisting of methyl, ethyl, propyl, isopropyl, butyl, s-butyl, isobutyl, pentyl, neopentyl, hexyl, heptyl, isoheptyl, 2-ethylhexyl, cyclohexyl and octyl;
the alkoxyl groups is selected from the group consisting of methoxyl, ethoxyl, propoxyl, isopropoxyl, butoxyl, s-butoxyl, isobutoxyl, pentoxyl, neopentoxyl, hexoxyl, heptoxyl, isoheptoxyl, 2-ethylhexoxyl and octoxyl;
the aryl is selected from phenyl, biphenyl, naphthyl, anthryl, phenanthryl, and heterocyclic compound having aromaticity which is select from furyl, thienyl, pyrryl, oxazolyl, thiazolyl, imidazolyl, pyrazolyl, or pyridyl;
“substituted” refers to the addition of one or more substituted groups to a compound or functional group, substituent groups do not cause the compounds to be unstable or unsuitable for use in an intended reaction and are inert under reaction conditions,
the substituent groups are selected from the group consisting of alkyl, alkoxyl, halogen, or nitrato.

7. The process according to claim 5, wherein the compounds for formula R₁—C(=O)—R₂ are cyclohexanone, acetophenone 4-methylacetophenone, 4-methoxyacetophenone, 4-nitroacetophenone, benzylacetone, 4-phenyl-3-buten-2-one, benzophenone and 4-chloroacetophenone and a mixture thereof;
the compounds for formula R₃—C(R₄)=N—R₅ are N-benzylideneaniline, N-(4-methylbenzylidene)aniline, 4-methyl-N-benzylideneaniline, N-(4-chlorobenzylidene)aniline, N-(4-methoxybenzylidene)aniline, N-(4-nitrobenzylidene)aniline or a mixture thereof.

8. The process according to claim 1, wherein the process is carried out at a temperature in the range of 20° C. to 60° C.;
the process is carried out in solvent selected from THF, glyme, diglyme, triglyme, crown ether, dichloromethane and dioxane;
the molar ratio of the metal amidoborane or derivatives to the compounds with polarized unsaturated functional group is 1:1 to 1:2;
the compounds for formula R₁—C(=O)—R₂ are reduced into corresponding secondary alcohols;
the compounds for formula R₃—C(R₄)=N—R₅ are reduced into corresponding secondary amines.

9. The process according to claim 1, wherein ligand Y is selected from NH₃BH₃, NH₃, pyridine, quadrol, triphenylphosphine and THF.
